(19) United States
(12) Patent Application Publication
(10) Pub. No.: US 2026/0232245 A1
(43) Pub. Date: Aug. 13, 2026

(54) IMPLANTABLE MEDICAL DEVICE WITH EMERGENCY COMMUNICATION MODE

(71) Applicant: Medtronic, Inc., Minneapolis, MN (US)

(72) Inventor: Paul G. Krause, Mahtomedi, MN (US)

(21) Appl. No.: 19/153,834

(22) PCT Filed: Jan. 2, 2024
(86) PCT No.: PCT/US2024/010063

§ 371 Date: Aug. 5, 2025

Related U.S. Application Data
(60) Provisional application No. 63/485,157, filed on Feb. 15, 2023.

(51) Int. Cl. A61B 5/29; A61B 5/00; A61B 5/318

(52) U.S. Cl. CPC A61B 5/29; A61B 5/0006; A61B 5/318; A61B 5/7275; A61B 5/7282; A61B 2560/0214

(57) ABSTRACT

An example device includes a sensing device to sense parameters of a patient; a first power source; a second power source; processing circuitry electrically coupled to the first power source and the second power source and configured to; collect, using power from the first power source, sensor data from tire sensing device; determine, using power from the first power source, whether the sensor data satisfies one or more acute health event criteria; in response to determining the sensor data does not satisfy the one or more acute health event criteria, continue to collect the sensor data from the sensing device using power from the first power source; and in response to determining the sensor data satisfies the one or more acute health event criteria, output, using power from the second power source, an indication of an acute health event to an external computing device using a far-field communication protocol.

This application is an international application with provisional priority of U.S. Provisional Patent Application No. 63/485,157, filed 15 Feb. 2023, the entire content of which is incorporated herein by reference.

### FIELD

The disclosure relates generally to a medical device and, more particularly, an implantable medical device configured to monitor patient parameters.

### BACKGROUND

A variety of devices are configured to monitor physiological parameters of a patient. Such devices include implantable or wearable medical devices, as well as a variety of wearable health or fitness tracking devices. The physiological parameters sensed by such devices may include as examples, electrocardiogram (ECG) signals, respiration signals, electroencephalogram (EEG) signals, perfusion signals, activity and/or posture signals, pressure signals, blood oxygen saturation signals, heart sounds signals, temperature signals, body composition signals, biochemical signals, fluid impedance signals, or blood constituent signals. There is a growing demand for using subcutaneous monitoring devices, which allow doctors to obtain information without a patient being connected to an external machine and/or which may otherwise not be reproducible in office settings.

### SUMMARY

Millions of patients worldwide are at an increased risk of an acute health event like cardiac arrest, stroke, syncopal (fainting) events, or other falls, yet few options exist for managing these patients. Implantable cardiac monitors provide useful data, but their use is limited by the burden required by cardiology clinics to review the data, and may not be used by some patients due to their size. Reducing a size of an implantable cardiac monitor may increase the number of patients willing to use it, but reducing the size of an implantable cardiac monitor may also reduce the size, longevity, and power capacity of a power source, such as a battery, of the implantable cardiac monitor. Wearable options are non-invasive and do not require cardiology clinics to review their data, but they may not be worn continuously due to patient discomfort and/or their frequent need of recharging and may not determine acute health events as accurately as an implantable medical device.

The techniques described herein are directed to a minimally invasive implantable medical device configured to communicate using near-field communication protocols during normal operation of the device, such as when collected sensor data does not satisfy one or more acute health event criteria, and configured to communicate using far-field communication protocols during an emergency operation of the device, such as when collected sensor data satisfies one or more acute health event criteria. The minimally invasive implantable medical device may include a first power source to provide power during normal operation of the device and a second power source (e.g., emergency power source) to provide power during emergency operation of the device, such as powering communication using far-field communication protocols during an emergency operation of the device.

Unlike conventional implantable medical devices and systems, the techniques and systems of this disclosure may provide reduced power source size of an implantable medical device while maintaining enough power to provide an indication that acute health events occurred. This combination may enable a size of an implantable medical device to be reduced, while also enabling the implantable medical device has sufficient power to send an indication of an acute health event using far-field communication protocols, such as during emergency situations. Additionally, as using the second power source may occur when a patient is suffering an acute health event, which may be unknown to them, it may be necessary for the implantable medical device to automatically switch to using the second power source to provide during an emergency operation of the device such as powering communication using far-field communication protocols during an emergency operation of the device. Using techniques of this disclosure in an implantable medical device may be advantageous when a physician cannot be continuously monitoring and evaluating the health event criteria to determine whether an acute health event criteria has been met and switch a power source to be used to provide power during emergency operation of the device.

In addition, the techniques and systems of this disclosure may be implemented in an implantable medical device that can continuously and/or periodically sense parameters of a patient without human intervention while subcutaneously implanted in a patient over months or years and perform millions of operations per second on patient sensor data to identify an acute health event. Using techniques of this disclosure in an implantable medical device may be advantageous when a physician cannot be continuously present with the patient over weeks or months to evaluate sensor data and/or where performing millions of operations on weeks or months of sensor data could not practically be performed in the mind of a physician with techniques of this disclosure.

In one example, a device comprises a sensing device to sense parameters of a patient; a first power source; a second power source; processing circuitry electrically coupled to the first power source and the second power source and configured to: collect, using power from the first power source, sensor data from the sensing device; determine, using power from the first power source, whether the sensor data satisfies one or more acute health event criteria; in response to determining the sensor data does not satisfy the one or more acute health event criteria, continue to collect the sensor data from the sensing device using power from the first power source; and in response to determining the sensor data satisfies the one or more acute health event criteria, output, using power from the second power source, an indication of an acute health event to an external computing device using a far-field communication protocol.

In another example, a method comprises collecting, by processing circuitry using power from a first power source, sensor data from a sensing device; determining, by the processing circuitry and using power from the first power source, whether the sensor data satisfies one or more acute health event criteria; in response to determining the sensor data does not satisfy the one or more acute health event criteria, continue collecting, by the processing circuitry using power from the first power source, sensor data from the sensing device; and in response to determining the sensor data satisfies the one or more acute health event criteria, outputting, by the processing circuitry via communication circuitry and using power from a second power source, an indication of an acute health event to an external computing device using a far-field communication protocol.

In another example, a non-transitory computer-readable storage medium comprising instructions that, when executed by processing circuitry, cause the processing circuitry to: collect, using power from a first power source, sensor data from a sensing device; determine, using power from the first power source, whether the sensor data satisfies one or more acute health event criteria; in response to determining the sensor data does not satisfy the one or more acute health event criteria, continue to collect, using power from the first power source, sensor data from the sensing device; and in response to determining the sensor data satisfies the one or more acute health event criteria, output, by the processing circuitry via communication circuitry and using power from a second power source, an indication of an acute health event to an external computing device using a far-field communication protocol.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a perspective view of an example implantable device in accordance with one or more techniques of this disclosure.

FIG. 2 is a block diagram illustrating an example configuration of an implantable device, in accordance with one or more techniques of this disclosure.

FIG. 3 is a perspective view of an example implantable device in accordance with one or more techniques of this disclosure.

FIG. 4 is a flow chart illustrating an example method to determine an acute health event of a patient and output an indication of the acute health event in accordance with one or more techniques of this disclosure.

### DETAILED DESCRIPTION

Various exemplary embodiments will now be described more fully with reference to the accompanying drawings in which some exemplary embodiments are illustrated. In the drawings, the thicknesses of layers and regions may be exaggerated for clarity.

Accordingly, while exemplary embodiments are capable of various modifications and alternative forms, embodiments thereof are shown by way of example in the drawings and will herein be described in detail. It should be understood, however, that there is no intent to limit exemplary embodiments to the particular forms disclosed, but on the contrary, exemplary embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of exemplary embodiments. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing only particular embodiments and is not intended to be limiting of exemplary embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which exemplary embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

The techniques described herein are directed to a minimally invasive implantable medical device, such as an injectable medical device, including an emergency power source configured to provide power to output signals via far-field communication protocols that may reach computing devices at a greater distance away from the patient but also require greater power levels. Thus, an implantable medical device may be small enough to be minimally invasive so more people may be willing to use the device, while also being able to output indications of an acute health event to a user's computing device and/or a computing device of someone other than the user, such as a nearby computing device (e.g., a smartphone of a person near patient experiencing an acute health event), an emergency technician computing device, a clinician computing device, and/or other computing device. This may conserve battery power in an implantable medical device that may enable reduced size of power sources in the implantable medical device. This may help provide a smaller implantable medical device to more patients, while also improving the reliability of the implantable medical device by helping to ensure an indication of an acute health event may be output to an external computing device using a far-field communication protocol so more immediate medical care may be provided to a patient having the implantable medical device when an acute health event occurs or is about to occur.

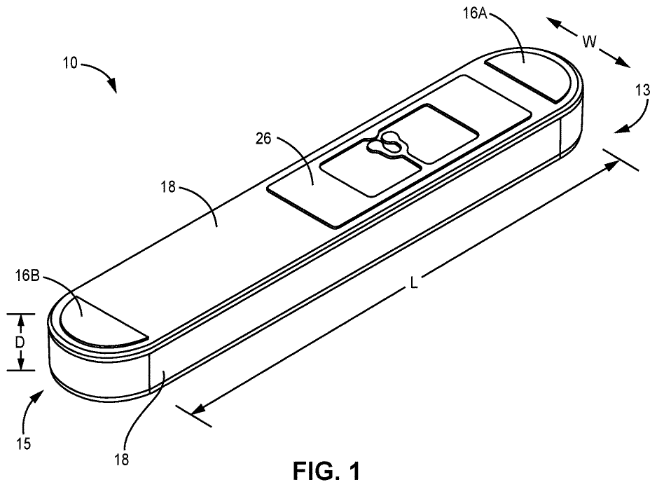

FIG. 1 shows an implantable medical device (IMD) 10 having two electrodes 16A and 16B (hereinafter “electrodes 16”), located adjacent the proximal end 13 and distal end 15, respectively, of IMD 10. In some examples, IMD 10 may be an injectable and/or insertable medical device. When implanted, such as by injection, electrodes 16, located on the upper surface 18 of the device may face outward toward the skin. In some examples, IMD 10 may have more than two electrodes 16. Electrodes 16 may additionally, or alternatively, be located on or extend to other surfaces of IMD 10. In the illustrated example, IMD 10 may include one or more antenna(s) 26 for wireless communication with other devices as described herein. Antenna 26 illustrated in FIG. 1 is a non-limiting example of antenna(s) included in IMD 10, as antenna(s) 26 may be of other shapes, sizes, or amounts than what is illustrated in the example shown in FIG. 1. For example, one or more antenna(s) 26 included in IMD 10 may be configured to perform near-field communication, while another one or more antennas of the antenna(s) 26 included in IMD 10 may be configured to perform far-field communication.

IMD 10, as an example illustrated in FIG. 1, may take the form of an elongated rectangular prism having rounded corners and a rounded distal end portion 15 as the rounded distal end of the device assists in allowing it to advance into body tissue, providing blunt dissection of the tissue as it advances. IMD 10 may have length (L), e.g., from proximal end 13 to distal end 15, width (W) and depth (D) as illustrated. In this particular embodiment, the width is greater than the depth, providing radial asymmetry along the longitudinal axis of the device and assisting in maintaining the IMD 10 in its proper orientation with upper surface 18 facing outward after being injected. In some examples, IMD 10, such as an elongated body of IMD, may take the form of a rounded and/or cylindrical shape, e.g., cross-sectional shape. In some examples, IMD 10 may be injected with other orientations as well. In some examples, IMD may include projections to prevent longitudinal and/or rotational movement of the device after being injected. In some examples, IMD 10 may have a length less than 5 centimeters (cm), a width less than 1 cm, a depth less than 0.5 cm, and a volume of less than 1.5 cubic centimeters (cm3). For example, IMD 10 may have a length of 45.1 millimeters (mm), a width of 8 mm, a depth of 4.2 mm, and a volume of 1.4cm3. In other examples, IMD 10 may have a length less than 46 mm, a width less than 4 mm, a depth less than 2 mm, and a volume of less than or equal to 0.25cm3. For example, IMD 10 may have a length from 30 mm to 45 mm, a width less than 4 mm, a depth less than 2 mm, and a volume of less than or equal to 0.25 cm3.

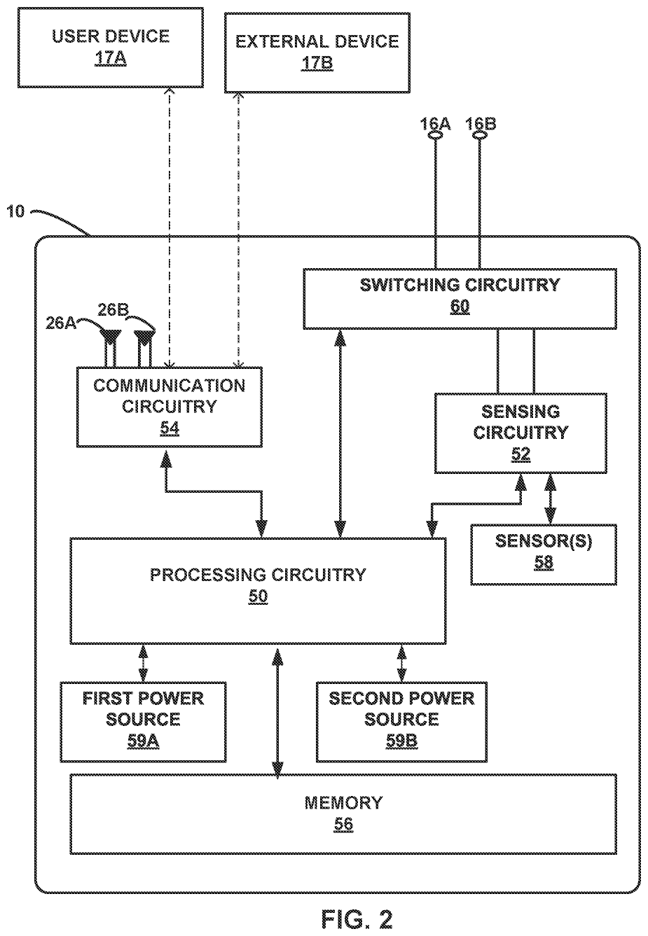

FIG. 2 is a block diagram illustrating an example configuration of IMD 10 of FIG. 1. As shown in FIG. 2, IMD 10 includes processing circuitry 50 sensing circuitry 52, communication circuitry 54, memory 56, sensor(s) 58, switching circuitry 60, first power source 59A, second power source 59B, and electrodes 16A and 16B, one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 50 may include fixed function circuitry and/or programmable processing circuitry. Processing circuitry 50 may include any one or more of a microprocessor, a controller, a digital signal processor (DSP), a graphical processing unit or tensor processing unit specialized for AI algorithm processing, an application specific integrated circuit (ASIC), a field-programmable gate array (FPGA), or equivalent discrete or analog logic circuitry, In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 60 as controlled by processing circuitry 50. Sensing circuitry 52 may monitor signals from electrodes 16 in order to monitor electrical activity of a heart of a patient and produce ECG data for patient. IMD 10 may include one or more sensors 58, such as one or more accelerometers, microphones, optical sensors, temperature sensors, biochemical sensors, and/or pressure sensors. Sensing circuitry 52 may include one or more filters and amplifiers for filtering and amplifying signals received from one or more of electrodes 16A, 16B and/or other sensors 58. In some examples, one or more of electrodes 16A, 16B and/or other sensors 58 may be referred to as a sensing device of IMD 10. In some examples, sensing circuitry 52 and/or processing circuitry 50 may include a rectifier, filter and/or amplifier, a sense amplifier, comparator, and/or analog-to-digital converter.

Sensing circuitry 52 and/or processing circuitry 50 may be configured to collect sensor data that may include one or more physiological parameters of patient based on signals from a sensing device, such as sensors 58 and/or electrodes 16. Some examples of physiological parameters include one or more of heart rate, respiratory rate, respiratory effort, fluid status, sympathetic tone, HRV, blood pressure, fluid redistribution, tissue perfusion, pulse oxygenation, sleep disordered breathing, heart sounds, ECG QRST morphology (R-wave amplitude, slope, width), EEG, biochemical sensor information, temperature, activity, and/or posture. Processing circuitry 50 may be configured to determine whether a patient is experiencing or just experienced an acute health event or determine an acute health event risk score of a likelihood the patient to experience an acute health event within a predetermined amount of time. Some examples of an acute health event include sudden cardiac arrest (SCA), stroke, heart attack, arrhythmias, myocardial infarction, heart failure decompensation, hypoglycemia, hyperglycemia, ketoacidosis, other metabolic disorders, or a fall.

Processing circuitry 50 may determine whether collected sensor data satisfies one or more acute health event criteria. In some examples, satisfaction of one or more acute health event criteria may indicate an acute health event is occurring or occurred in patient or an acute health event risk score of patient is greater than or equal to an emergency threshold. In some examples, IMD 10 may determine the acute health event and/or an acute health event risk score in accordance with U.S. application Ser. Nos. 12/184,149 and 12/184,003 by Sarkar et al., entitled “USING MULTIPLE DIAGNOSTIC PARAMETERS FOR PREDICTING HEART FAILURE EVENTS,” and “DETECTING WORSENING HEART FAILURE BASED ON IMPEDANCE MEASUREMENTS,” both filed on Jul. 31, 2008, U.S. application Ser. No. 16/940,817 by Bumes et al., entitled “DETERMINING A FALL RISK RESPONSIVE TO DETECTING BODY POSITION MOVEMENTS,” filed Jul. 28, 2020, and/or U.S. application Ser. No. 17/246,331 by Cho et. al., entitled “ACUTE HEALTH EVENT MONITORING,” filed Apr. 30, 2021, all of which are incorporated herein by reference in their entirety.

In some examples, to determine whether collected sensor data satisfies one or more acute health event criteria, processing circuitry may apply a set of rules to the sensor data to determine a risk score, determine whether the risk score satisfies an acute health event threshold, and in response to determining the risk score satisfies an acute health event threshold, determine the sensor data satisfies one or more acute health event criteria.

Processing circuitry 50 may compare the determined acute health event risk score to an emergency threshold. In some examples, the determined acute health event risk score being greater than or equal to the emergency threshold indicates a patient is highly likely to experience an acute health event in a short period of time, such as within the next 24 hours, within the next 12 hours, within the next 6 hours, or within the next I hour, and will need to seek immediate medical attention. In some examples, the short period of time may be longer than 24 hours, shorter than 1 hour, or anywhere between 1 hour and 24 hours.

A patient having collected sensor data satisfying one or more acute health event criteria, such as a patient that is experiencing or experiences an acute health event or is about to experience an acute health event, may need immediate medical attention to reduce potential harm that may be caused by the acute health event and/or to save the life of a patient experiencing, just experienced, or about to experience the acute health event. In some examples, processing circuitry 50 determining collected sensor data does not satisfy one or more acute health event criteria, such as an acute health event did not occur or that an acute health event risk score is less than an emergency threshold, may be referred to as “normal operation” of IMD 10. Processing circuitry 50 determining collected sensor data satisfies one or more acute health event criteria, such as an acute health event did occur or is occurring or that an acute health event risk score is greater than or equal to an emergency threshold, may be referred to as “emergency operation” of IMD 10.

Communication circuitry 54 may include any suitable hardware, firmware, software or any combination thereof for communicating with another device, such as external device 12. Communication circuitry 54 may be configured to communicate using any of a variety of wireless communication schemes, such as near-field communication technologies (e.g., inductive coupling, Near-Field Communication (NFC) or other communication technologies operable at ranges less than 10-20 centimeters (cm)) and/or far-field communication technologies (e.g., cellular (e.g., 3G, 4G, 5G), Wi-Fi (e.g., 802.11 communication protocol or 802.15 ZigBee communication protocol), short-range wireless (e.g., Bluetooth® communication protocol or Bluetooth® Low Energy (BLE) communication protocol, satellite communication (e.g., Starlink), or other communication technologies operable at ranges greater than near-field communication technologies). In some examples, near-field communication technologies may communicate within a wireless range of the communication protocol ISO/IEC 14443. In some examples the near-field communication technologies may have a range of 4 centimeters or less. In some examples, under the control of processing circuitry 50, communication circuitry 54 may receive downlink telemetry from, as well as send uplink telemetry to, user device 17A, external device 17B or another device with the aid of an internal or external antenna, e.g., antenna(s) 26. IMD 10 may include one or more antennas 26. For example, as illustrated in FIG. 2, IMD 10 may include two antennas 26A, 26B. In some examples, IMD 10 may have one antenna to provide the communication capabilities of antennas 26A, 26B. In some examples, IMD 10 may have more than two antennas.

Communication circuitry 54 communicating using near-field communication technologies or protocols requires less power and battery capacity than communicating using far-field communication technologies or protocols. Communication circuitry 54 may be configured to use power, if necessary, from first power source 59A to power communication using near-field communication technologies or protocols. Communication circuitry 54 may be configured to use power from second power source 59B to power communication using far-field communication technologies or protocols. Communication circuitry 54 may communicate using near-field communication technologies or protocols via antenna 26A and may communicate using far-field communication technologies or protocols via antenna 26B. In some examples, communication circuitry 54 may include two separate circuitries, a first communication circuitry to communicate using near-field communication technologies or protocols via antenna 26A, and a second communication circuitry to communicate using far-field communication technologies or protocols via antenna 26B. For examples, antenna 26A may be optimized to communicate using near-field communication technologies or protocols and antenna 26B may be optimized to communicate using far-field communication technologies or protocols. In some examples, communication circuitry 54 may communicate using near-field communication technologies or protocols and communicate using far-field communication technologies or protocols via the same antenna.

During normal operations of IMD 10, first power source 59A is configured to provide power to various components of IMD 10, such as processing circuitry 50, sensing circuitry 52, and communication circuitry 54. In addition, circuitry, such as sensing circuitry 52 and processing circuitry 50 are configured to use power from the first power source 59A to perform collecting sensor data and determining of whether the collected sensor data satisfies one or more acute health event criteria.

During normal operations of IMD 10, IMD 10 may send the sensor data to user device 17A using near-field communication. In some examples, IMD 10 may send the sensor data to user device 17A using near-field communication via antenna 26A, which is optimized to communicate using near-field communication technologies or protocols. In some examples, in response to determining the collected sensor data did not satisfy one or more acute health event criteria., processing circuitry 50 may continue to collect sensor data. In addition, processing circuitry 50 may be and/or remain configured to transmit, via communication circuitry 54, the sensor data to user device 17A using near-field communication. For example, a patient may place user device 17A close to the IMD 10, such as within 10 cm. When a user device 17A gets within a near-field communication threshold, for example, within 5 or 10 cm, user device 17A may initiate IMD 10 to output sensor data to user device 17A. For example, user device 17A may send a signal to IMD 10 to output sensor data to user device 17A. In response to receiving the signal from user device 17A, IMD 10 may output, such as via processing circuitry 50 and communication circuitry 54, sensor data to user device 17A. User device 17A may display the collected sensor data on a display of the user device 17A. In some examples, user device 17A may send the collected sensor data to another device, such as a clinician computing device, cloud storage, or a cloud-based platform.

In some examples, near-field communication may not use power from first power source 59A or may use minimal power from first power source 59A. For example, processing circuitry 50 may transmit sensor data to user device 17A using near-field communication by using power from first power source 59A or not using power from any power source in IMD 10. For example, IMD 10 may receive an NFC pulse from user device 17A that energizes a capacitor, first power source 59A, or other small battery in IMD 10, and that energy received from the NFC pulse is then used to transmit sensor data to user device 17A. In some examples, since first power source 59A may be used for processing operations of IMD 10 and not for communication operations using a far-field communication protocol, a size (e.g., volume) and/or weight of first power source 59A may be minimized to reduce size and/or weight of first power source 59A in IMD 10. In some examples, first power source 59A may be a battery. For example, first power source 59A may be comprised of a Li-SVO/CFx chemistry, such as a lithium anode and silver vanadium oxide and fluorinated carbon cathode. In some examples, the volume of first power source 59A is between 50%-60% of the IMD 10 volume. In other examples, the volume of first power source 59A may be less than or equal to 50% of the IMD 10 volume. In other examples, the volume of first power source 59A may be greater than or equal to 60% of the IMD 10 volume. In some examples, the electrical capacity of first power source 59A may be between 100 milliamp hour (mAh) to 200 mAh. In other examples, the electrical capacity of first power source 59A may be less than or equal to 100 mAh. In other examples, the electrical capacity of first power source 59A may be greater than or equal to 200 mAh.

In response to determining collected sensor data satisfies one or more acute health event criteria, such as an acute health event occurred or is occurring or the acute health event risk score being greater than or equal to the emergency threshold, processing circuitry 50 may transmit, via communication circuitry 54, an indication of an acute health event to an external computing device 17B using a far-field communication protocol. In some examples, in response to determining collected sensor data satisfies one or more acute health event criteria, processing circuitry 50 may transmit, via communication circuitry 54, advertisements for external devices. In some examples, external computing device 17B may have larger power resources compared to IMD 10 and be configured to listen to advertisements. For examples, external computing device 17B may be configured to continually listen for advertisements. Upon receiving an advertisement from IMD 10, external computing device 17B may communicatively couple to IMD 10, and then processing circuitry 50 may transmit, via communication circuitry 54, an acute health event to an external computing device 17B using a far-field communication protocol.

Processing circuitry 50 may transmit, via communication circuitry 54, the indication using power from second power source 59B. In some examples, the second power source 59B may be reserved for emergency transmissions that may require immediate medical attention for patient, such as sending an indication of an acute health event to external computing device 17B. Accordingly, IMD 10 would have enough battery power to transmit the indication using a far-field communication protocol, which uses more power than near-field communication.

In some examples, second power source 59B may be a small primary cell battery or a small rechargeable battery. In other examples, second power source 59B may be a capacitor or a “super capacitor”. In some examples, the capacity of second power source 59B may be enough to supply one or two emergency transmissions to external computing device 17B. In some examples, capacity of second power source 59B may be less than capacity of first power source 59A. In some examples, capacity of second power source 59B may less than 10 mAh. In some examples, capacity of second power source 59B may be between 1 mAh and 2 mAh. In other examples, second power source 59B may have a capacity different than those described above, such as a capacity greater than 10 mAh.

In examples in which second power source 59B is a rechargeable battery or a capacitor, second power source 59B may be recharged, to supply additional emergency transmissions for future events. In some examples, recharging may take place using an external recharger or from first power source 59A. In examples in which second power source 59B is a primary cell battery, additional emergency transmissions would not be possible once second power source 59B battery capacity has been consumed.

For example, second power source 59B may include enough energy to be used for sending an indication of an acute health event during one period of time and need to be replaced after use. In some examples, second power source 59B may need to be recharged after sending an indication of an acute health event. In some examples, second power source 59B may include enough energy to be used for sending an indication of an acute health event for multiple occurrences of a health event. In some examples, since second power source 59B may be reserved for sending an indication of an acute health event using far-field communication protocol a size and/or weight of second power source 59B may be minimized to reduce size and/or weight of first power source 59B in IMD 10.

IMD 10 having a reduced size, as described above, limits the size of the power source(s) that may be positioned in IMD 10. Since IMD 10 may reserve communicating via a far-field communication protocol for emergency situations, power source 59A, 59B size may be reduced in IMD 10, such as described above, which reduces size of IMD 10 and makes implantation of IMD 10 less invasive. In addition, since IMD 10 has two separate power sources 59A, 59B, and one power source (e.g., second power source 59B) designated specifically for providing power for sending an indication of an acute health event using a far-field communication protocol, reliability of IMD 10 having enough power to be able to transmit an indication of an acute health event during an emergency may be improved. IMD 10 having a combination of a reduced size, that may make it less invasive, while maintaining or improving reliability of transmissions during an emergency event may help IMD 10 to be used by more patients, which may lead to faster and better treatment that may improve health/treatment of people experiencing, just experienced, or about to experience an acute health event. In addition, determining and indicating an occurrence of an acute health event upon it occurring or when it is about to occur, may also reduce treatment costs.

In some examples, user device 17A and external device 17B may be the same device. In some examples, user device 17A and external device 17B may be separate devices. For example, user device 17A may be a smartphone, tablet, smartwatch, or other computing device of a patient and external device 17B may be a smartphone, tablet, smartwatch, or other computing device of someone besides the patient, such as a clinician, emergency technician, or another device in the near vicinity of IMD 10 that is not a device of the patient.

In some examples, when user device 17A and external device 17B are the same device, such as a smartphone or smartwatch, a patient may be able to retrieve sensor data during normal operation of IMD 10. For example, patient may “tap” the user device 17A to an area where IMD 10 was implanted when patient desires to review the collected sensor data. When patient taps user device 17A, IMD 10 communicates with user device 17A using near-field communication protocols. This allows patient to retrieve sensor data when desired while minimizing power usage. When the collected sensor data satisfies one or more acute health event criteria, IMD communicates with user device 17A using far-field communication protocols that provide transmission distances greater than near-field communication protocols. For example, a patient having sensor data satisfying one or more acute health event criteria may be unable or have difficulty bringing user device 17A within range of IMD 10 for near-field communication protocols to work. This may enable user device 17A to receive an indication of an acute health event during an emergency, which may enable a patient to obtain treatment sooner.

In some examples, upon receiving an acute health event indication from IMD 10, user device 17A may output an alarm that may be visual and/or audible, and configured to immediately attract the attention of patient or any person in environment with patient, e.g., a bystander. In some examples, upon receiving an acute health event indication from IMD 10, user device 17A may send an indication of an acute health event to an external device 17B, such as a health monitoring system (HMS), cloud service, one or more Internet of Things (IoT) devices. IoT devices may include, as examples, so called “smart” speakers, cameras, televisions, lights, locks, thermostats, appliances, actuators, controllers, or any other smart home (or building) devices. IoT devices may provide audible and/or visual alarms when configured with output devices to do so. As other examples, IoT devices may cause smart lights throughout environment to flash or blink and unlock doors. In some examples, IoT devices that include cameras or other sensors may activate those sensors to collect data regarding patient, e.g., for evaluation of the condition of patient. In some examples, IMD 10 may output an indication of an acute health event directly to an external device 17B. In some examples, IMD 10 may output an indication of an acute health event to an external device 17B and user device 17A.

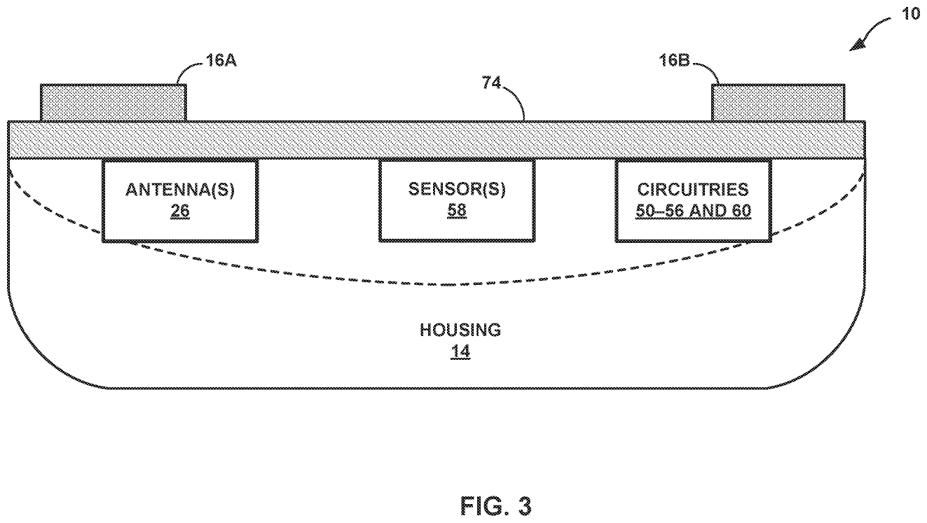

FIG. 3 is a conceptual side-view diagram illustrating an example configuration of IMD 10. In the example shown in FIG. 3, IMD 10 may include a leadless, subcutaneously-injectable monitoring device having a housing 14 and an insulative cover 74. Electrode 16A and electrode 16B may be formed or placed on an outer surface of cover 74. Circuitries 50-56 and 60, described above with respect to FIG. 2, may be formed or placed on an inner surface of cover 74, or within housing 14. In the illustrated example, antenna(s) 26 is formed or placed on the inner surface of cover 74, but may be formed or placed on the outer surface in some examples. Sensors 58 may also be formed or placed on the inner or outer surface of cover 74 in some examples. In some examples, insulative cover 74 may be positioned over an open housing 14 such that housing 14 and cover 74 enclose antenna(s) 26, sensors 58, and circuitries 50-56 and 60, and protect the antenna and circuitries from fluids such as body fluids.

One or more of antenna(s) 26, sensors 58, or circuitries 50-56 may be formed on insulative cover 74, such as by using flip-chip technology. In some examples, an insulating material may be sprayed onto the outer housing 14, except for electrodes 16, to form an insulative cover to contact patient, such as body fluids from patient. In some examples, housing 14 may enclose the entire IMD 10. In some examples, housing 14, such as titanium, may partially enclose IMD 10 while another biocompatible material, such as glass, sapphire, etc., may enclose the remaining portion of IMD 10. Electrodes 16 may be electrically connected to switching circuitry 60 through one or more vias (not shown) formed through insulative cover 74. Insulative cover 74 may be formed of sapphire (i.e., corundum), glass, parylene, and/or any other suitable insulating material. Housing 14 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

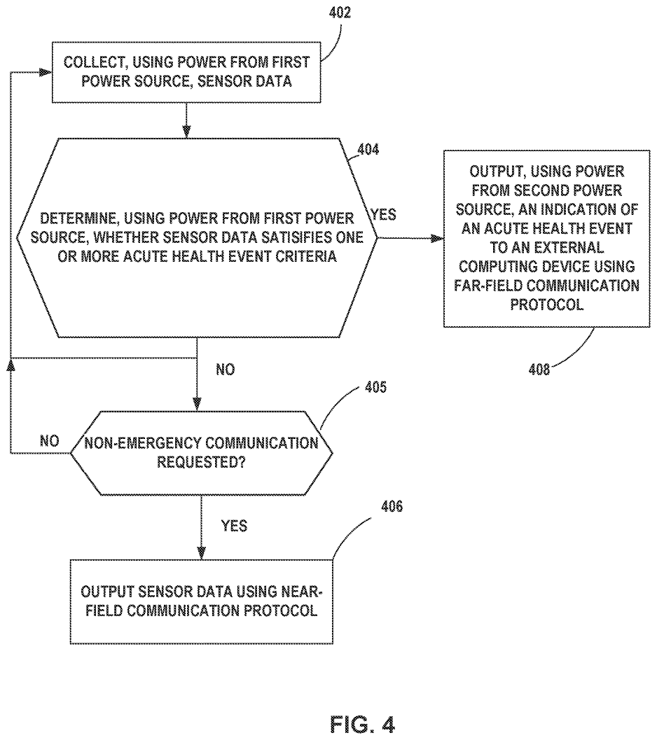

FIG. 4 is a flow chart illustrating a preferred embodiment of monitoring and reporting an acute health event, according to the present invention. Processing circuitry 50 may collect, using power from first power source, sensor data (402). Processing circuitry 50 may determine, using power from first power source, whether sensor data satisfies one or more acute health event criteria (404). In response to determining sensor data does not satisfy one or more acute health event criteria, processing circuitry 50 may continue normal operation of collecting sensor data (402) or determine whether non-emergency communication is requested (405). In response to determining non-emergency communication is requested, such as by user device 17A, processing circuitry 50 outputs via communication circuitry 54 sensor data using near-field communication protocols (406). In response to determining sensor data satisfies one or more acute health event criteria, processing circuitry 50 outputs via communication circuitry 54, using power from second power source, an indication of an acute health event to an external computing device using far-field communication protocols (408).

The following examples are illustrative of the techniques described herein.

Example 1: A device includes a sensing device to sense parameters of a patient; a first power source; a second power source; processing circuitry electrically coupled to the first power source and the second power source and configured to: collect, using power from the first power source, sensor data from the sensing device; determine, using power from the first power source, whether the sensor data satisfies one or more acute health event criteria; in response to determining the sensor data does not satisfy the one or more acute health event criteria, continue to collect the sensor data from the sensing device using power from the first power source; and in response to determining the sensor data satisfies the one or more acute health event criteria, output, using power from the second power source, an indication of an acute health event to an external computing device using a far-field communication protocol.

Example 2: The device of example 1, wherein the processing circuitry is further configured to output the sensor data to a user computing device using a near-field communication protocol.

Example 3: The device of example 2, wherein the processing circuitry is configured to output the sensor data to the user computing device using the near-field communication protocol using power from the first power source, Example 4: The device of any of examples 1-3, wherein in response to determining the sensor data satisfies the one or more acute health event criteria, the processing circuitry is further configured to output, using power from the second power source and using the far-field communication protocol, treatment adjustment instructions to the external computing device.

Example 5: The device of any of examples 1-4, wherein the device is configured to be injected in the patient.

Example 6: The device of any of examples 2-5, wherein the user device and the external computing device are the same device.

Example 7: The device of any of examples 2-6, wherein the user device is a smartphone.

Example 8: The device of any of examples 1-7, wherein the device comprises a length less than 5 centimeters (cm), a width less than 1 cm, and a depth less than 0.5 cm.

Example 9: The device of any of examples 1-8, wherein the device is an insertable cardiac monitor (ICM), the sensing device comprises two electrodes, and the ICM is configured to monitor an electrocardiogram of a patient via the two electrodes.

Example 10: The device of any of examples 1-9, wherein the acute health event is a sudden cardiac arrest, myocardial infarction, arrhythmia, heart failure decompensation, hypoglycemia, hyperglycemia, ketoacidosis, a stroke, or a fall.

Example 11: The device of any of examples 1-10, wherein the far-field communication protocol is short-range wireless, cellular, satellite, or Wi-Fi.

Example 12: The device of any of examples 1-11, wherein a volume of the device is less than or equal to 0.25 cubic centimeters.

Example 13: The device of any of examples 1-12, wherein the acute health event criteria comprises an acute health event risk score being greater than or equal to an emergency threshold, an acute event occurred, or an acute health event is occurring.

Example 14: The device of any of examples 2-13, wherein the processing circuitry is further configured to: receive a signal, from the user device, to output the sensor data to the user device; and in response to receiving the signal from the user device, output the sensor data to the user computing device using the near-field communication protocol.

Example 15: The device of any of examples 1-14, wherein the processing circuitry is further configured to: in response to determining the sensor data satisfies the one or more acute health event criteria, output an advertisement to communicatively couple the processing circuitry with the external computing device; in response to processing circuitry communicatively coupling with the external computing device, output the indication of the acute health event to the external computing device using the far-field communication protocol.

Example 16: The device of any of examples 1-15, wherein to determine, using power from the first power source, whether the sensor data satisfies one or more acute health event criteria the processing circuitry is further configured to: apply a set of rules to the sensor data to determine a risk score; determine whether the risk score satisfies an acute health event threshold; and in response to determining the risk score satisfies an acute health event threshold, determine the sensor data satisfies one or more acute health event criteria.

Example 17: A method includes collecting, by processing circuitry using power from a first power source, sensor data from a sensing device; determining, by the processing circuitry and using power from the first power source, whether the sensor data satisfies one or more acute health event criteria; in response to determining the sensor data does not satisfy the one or more acute health event criteria, continue collecting, by the processing circuitry using power from the first power source, sensor data from the sensing device; and in response to determining the sensor data satisfies the one or more acute health event criteria, outputting, by the processing circuitry via communication circuitry and using power from a second power source, an indication of an acute health event to an external computing device using a far-field communication protocol.

Example 18: The method of example 17, further includes outputting the sensor data to a user computing device using a near-field communication protocol.

Example 19: The method of example 18, further includes outputting the sensor data to a user computing device using a near-field communication protocol using power from the first power source.

Example 20: The method of any of examples 17-19, further includes in response to determining the sensor data satisfies the one or more acute health event criteria, outputting, using power from the second power source and using the far-field communication protocol, treatment adjustment instructions to the external computing device.

Example 21; The method of any of examples 18-20, wherein the user device and the external computing device are the same device.

Example 22: The method of any of examples 18-20, wherein the user device is a smartphone.

Example 23: The method of any of examples 17-22, wherein the acute health event is a sudden cardiac arrest, myocardial infarction, hypoglycemia, a stroke, or a fall.

Example 24: The method of any of examples 17-23, wherein the far-field communication protocol is short-range wireless, cellular, satellite, or Wi-Fi, Example 25: The method of any of examples 17-24, wherein the acute health event criteria comprises an acute health event risk score being greater than or equal to an emergency threshold, an acute event occurred, or an acute health event is occurring.

Example 26: The method of any of examples 18-25, further includes receiving a signal, from the user device, to output the sensor data to the user device; and in response to receiving the signal from the user device, outputting the sensor data to the user computing device using the near-field communication protocol.

Example 27: The method of any of examples 18-26, further includes in response to determining the sensor data satisfies the one or more acute health event criteria, outputting an advertisement to communicatively couple the processing circuitry with the external computing device; in response to processing circuitry communicatively coupling with the external computing device, outputting the indication of the acute health event to the external computing device using the far-field communication protocol.

Example 28: The method of any of examples 18-27, wherein determining, by the processing circuitry and using power from the first power source, whether the sensor data satisfies one or more acute health event criteria comprises: applying a set of rules to the sensor data to determine a risk score; determining whether the risk score satisfies an acute health event threshold; and in response to determining the risk score satisfies an acute health event threshold, determine the sensor data satisfies one or more acute health event criteria.

Example 29: A non-transitory computer-readable storage medium includes collect, using power from a first power source, sensor data from a sensing device; determine, using power from the first power source, whether the sensor data satisfies one or more acute health event criteria; in response to determining the sensor data does not satisfy the one or more acute health event criteria, continue to collect, using power from the first power source, sensor data from the sensing device; and in response to determining the sensor data satisfies the one or more acute health event criteria, output, by the processing circuitry via communication circuitry and using power from a second power source, an indication of an acute health event to an external computing device using a far-field communication protocol.

Example 30: The non-transitory computer-readable storage medium of example 29, wherein the processing circuitry is further caused to: output the sensor data to a user computing device using a near-field communication protocol using power from the first power source.

Example 31: The non-transitory computer-readable storage medium of any of examples 29-30, wherein the processing circuitry is further caused to: in response to determining the sensor data satisfies the one or more acute health event criteria, output, using power from the second power source and using the far-field communication protocol, treatment adjustment instructions to the external computing device based on the determined acute health event risk score or the determined acute health event.

Various examples have been described. These and other examples are within the scope of the following claims.

1. A device comprising:
a sensing device to sense parameters of a patient;
a first power source;
a second power source;
processing circuitry electrically coupled to the first power source and the second power source and configured to:
collect, using power from the first power source, sensor data from the sensing device;
determine, using power from the first power source, whether the sensor data satisfies one or more acute health event criteria;
in response to determining the sensor data does not satisfy the one or more acute health event criteria, continue to collect the sensor data from the sensing device using power from the first power source; and
in response to determining the sensor data satisfies the one or more acute health event criteria, output, using power from the second power source, an indication of an acute health event to an external computing device using a far-field communication protocol,
wherein the processing circuitry is configured to only use power from the second power source in response to determining the sensor data satisfies the one or more acute health event criteria.

2. The device of claim 1, wherein the processing circuitry is further configured to output the sensor data to a user computing device using a near-field communication protocol.

3. The device of claim 2, wherein the processing circuitry is configured to output the sensor data to the user computing device using the near-field communication protocol using power from the first power source.

4. The device of claim 1, wherein in response to determining the sensor data satisfies the one or more acute health event criteria, the processing circuitry is further configured to output, using power from the second power source and using the far-field communication protocol, treatment adjustment instructions to the external computing device.

5. The device of claim 1, wherein the device is configured to be injected in the patient.

6. The device of claim 2, wherein the user computing device and the external computing device are the same device.

7. The device of claim 1, wherein the device comprises a length less than 5 centimeters (cm), a width less than 1 cm, and a depth less than 0.5 cm.

8. The device of claim 1, wherein the device is an insertable cardiac monitor (ICM), the sensing device comprises two electrodes, and the ICM is configured to monitor an electrocardiogram of a patient via the two electrodes.

9. The device of claim 1, wherein the acute health event is a sudden cardiac arrest, myocardial infarction, arrhythmia, heart failure decompensation, hypoglycemia, hyperglycemia, ketoacidosis, a stroke, or a fall.

10. The device of claim 1, wherein the far-field communication protocol is short-range wireless, cellular, satellite, or Wi-Fi.

11. The device of claim 1, wherein a volume of the device is less than or equal to 0.25 cubic centimeters.

12. The device of claim 1, wherein the acute health event criteria comprises an acute health event risk score being greater than or equal to an emergency threshold, an acute event occurred, or an acute health event is occurring.

13. The device of claim 2, wherein the processing circuitry is further configured to:
receive a signal, from the user device, to output the sensor data to the user device; and
in response to receiving the signal from the user device, output the sensor data to the user computing device using the near-field communication protocol.

14. The device of claim 1, wherein the processing circuitry is further configured to:
in response to determining the sensor data satisfies the one or more acute health event criteria, output an advertisement to communicatively couple the processing circuitry with the external computing device; and
in response to processing circuitry communicatively coupling with the external computing device, output the indication of the acute health event to the external computing device using the far-field communication protocol.

15. The device of claim 4, wherein to determine, using power from the first power source, whether the sensor data satisfies one or more acute health event criteria the processing circuitry is further configured to:
apply a set of rules to the sensor data to determine a risk score;
determine whether the risk score satisfies an acute health event threshold; and
in response to determining the risk score satisfies an acute health event threshold, determine the sensor data satisfies one or more acute health event criteria.

16. A non-transitory computer-readable storage medium comprising instructions that, when executed by processing circuitry, cause the processing circuitry to:
collect, using power from a first power source, sensor data from a sensing device;
determine, using power from the first power source, whether the sensor data satisfies one or more acute health event criteria;
in response to determining the sensor data does not satisfy the one or more acute health event criteria, continue to collect, using power from the first power source, sensor data from the sensing device; and
in response to determining the sensor data satisfies the one or more acute health event criteria, output, by the processing circuitry via communication circuitry and using power from a second power source, an indication of an acute health event to an external computing device using a far-field communication protocol,
wherein the processing circuitry is configured to only use power from the second power source in response to determining the sensor data satisfies the one or more acute health event criteria.

17. A method comprising:
collecting, by processing circuitry using power from a first power source, sensor data from a sensing device;
determining, by the processing circuitry and using power from the first power source, whether the sensor data satisfies one or more acute health event criteria;
in response to determining the sensor data does not satisfy the one or more acute health event criteria, continue collecting, by the processing circuitry using power from the first power source, sensor data from the sensing device; and
in response to determining the sensor data satisfies the one or more acute health event criteria, outputting, by the processing circuitry via communication circuitry and only using power from a second power source, an indication of an acute health event to an external computing device using a far-field communication protocol.

18. The method of claim 17, further comprising:
outputting the sensor data to a user computing device using a near-field communication protocol using power from the first power source.

19. The method of claim 18, further comprising:
in response to determining the sensor data satisfies the one or more acute health event criteria, outputting, using power from the second power source and using the far-field communication protocol, treatment adjustment instructions to the external computing device.

20. The method of claim 17, further comprising:
in response to determining the sensor data satisfies the one or more acute health event criteria, outputting an advertisement to communicatively couple the processing circuitry with the external computing device;
in response to processing circuitry communicatively coupling with the external computing device, outputting the indication of the acute health event to the external computing device using the far-field communication protocol.
